(19) United States
(12) Patent Application Publication
(10) Pub. No.: US 2016/0183563 A1
(43) Pub. Date: Jun. 30, 2016

(54) FEED FOR LACTATING RUMINANTS

(71) Applicant: BENEMILK OY, Raisio (FI)

(72) Inventors: Feng Wan, Issaquah, WA (US); James Edward Nocek, Auburn, NY (US); Merja Birgitta Holma, Raisio (FI); Timothy Martin Londergan, Seattle, WA (US); Ilmo Pellervo Aronen, Hinnerjoki (FI); Illimar Altosaar, Ottawa (CA); Craig Cano Beeson, Charleston, SC (US); Jayesh Ramesh Bellare, Mumbai (IN)

(73) Assignee: Benemilk Oy, Raisio (FI)

(21) Appl. No.: 14/908,108

(22) PCT Filed: Jul. 30, 2013
(86) PCT No.: PCT/US2013/052655

§ 371 Date: Jan. 27, 2016

(51) Int. Cl. A23K 1/16; A61K 36/899; A61K 36/21; A61K 31/197; A61K 33/42; A61K 33/10; A61K 33/24; A61K 33/08; A61K 33/32; A61K 33/30; A61K 33/26; A61K 31/28; A61K 31/295; A61K 31/315; A61K 47/48; A61K 31/20; A61K 31/202

(52) U.S. Cl. CPC A23K 1/1813; A23K 1/164; A23K 1/1603; A23K 1/1758; A23K 1/1751; A23K 1/1753; A61K 47/4823; A61K 31/20; A61K 47/48246; A61K 36/899; A61K 36/21; A61K 31/197; A61K 33/42; A61K 33/10; A61K 33/24; A61K 33/08; A61K 33/32; A61K 33/30; A61K 33/26; A61K 31/28; A61K 31/295; A61K 31/315

(57) ABSTRACT

A feed for ruminants may include at least one fatty acid component covalently linked with a carrier particle such that ingestion of the feed by lactating ruminants may provide for an increase in the amount of milk produced by the ruminant, and/or an increase in the fat content of the milk produced.

### BACKGROUND

Increasing milk production and improving milk quality have always been a major goal when feeding lactating dairy animals, such as dairy cows. Depending on the animal, the feed components may vary considerably. For example, ruminants are able to digest fibrous plant based foods, or roughage, that are indigestible to non-ruminants. Ruminants may include lactating animals such as, for example, cattle, goats, sheep, and dairy cows. Some examples of roughages include hay, grass silage, corn silage, straw and pasture, as well as various whole grain/leguminous silages and other fodders.

For efficient milk production, ruminants may also be given, in addition to roughages, a feed concentrate that may include energy components (that is, carbohydrates and fat), protein components, minerals, micronutrients and vitamins. Some examples of common feed items include grain feeds (corn, oats, barley, wheat), vegetable oilseed crushes or meal (rapeseed) and soybeans. A large variety of different byproducts from food industry may also be used.

Although there has been an increase in the milk production of cows during the last decades, the degree of utilization of feed has essentially not improved. The same amount of energy intake per kilogram of milk is needed now as was needed decades ago. When the utilization of energy becomes more effective, milk production may increase and the concentration of protein and fat in the milk may increase.

Most attempts for increasing milk fat content tend to lower milk production and/or protein content, and result in other undesired effects, such as increased trans fatty acid levels, on the fatty acid profile of the milk fat. Therefore, there still remains a need for new compositions and methods that can increase production of milk with increased levels of milk fat.

### SUMMARY

In an embodiment a feed composition for ruminants includes at least one carrier, at least one fatty acid moiety covalently linked to the carrier, and at least one nutritional component.

In an embodiment, a nutriment for ruminants includes an ingestible polymer carrier, and at least one palmitic acid moiety covalently linked to the polymer carrier.

In an embodiment, a method for producing a nutriment for ruminants includes covalently bonding at least one palmitic acid moiety to a carrier to produce palmitic acid particles, and dispersing the palmitic acid particles in at least one of ruminant feed and drinking water for ingestion by the ruminant.

In an embodiment, a method for increasing at least one of an amount of milk produced by a lactating ruminant and a milk fat content in the milk produced by the lactating ruminant includes feeding the lactating ruminant a feed composition having at least one palmitic acid moiety covalently bound to a carrier.

### BRIEF DESCRIPTION OF THE FIGURES

FIG. 1 depicts a representation of a carrier particle with palmitic acid molecules covalently attached according to an embodiment.

FIG. 2 depicts a simplified hemi-cellulose particle with covalently linked palmitic acid according to an embodiment.

### DETAILED DESCRIPTION

With relation to the description as presented herein, a “ruminant” is a class of mammal with a multiple chamber stomach that gives the animal an ability to digest cellulose-based food by softening it within the first chamber (rumen) of the stomach and regurgitating the semi-digested mass. The regurgitate, known as cud, is then chewed again by the ruminant. Specific examples of ruminants include, but are not limited to, cattle, bison, buffaloes, yaks, camels, llamas, giraffes, deer, pronghorns, antelopes, sheep, and goats. The milk produced by ruminants is widely used in a variety of dairy-based products. Dairy cows are of considerable commercial significance for the production of milk and processed dairy products such as, for example, yogurt, cheese, whey, and ice cream.

The formation of milk in the mammary gland is a complex enzymatic process regulated by hormones, requiring lots of ATP energy at the cell level, as well as suitable starting materials and enzymes. The main components of milk, that is, lactose, protein, and fat, are synthesized in the cells of the udder. Glucose availability in the mammary gland has been regarded as the main limiting factor in milk production, in addition to the availability of some amino acids. Acetate is also an important starting material of de novo synthesis of milk fat. Acetate provides a relevant source of energy, and acetate has been determined to have a unique role in energy metabolism as part of ATP formation in the synthesis of all milk components.

In feeding of ruminants, as mentioned above, roughages may be supplemented with energy and protein components, minerals, micronutrients and vitamins, etc. The inclusion of fat has been generally minimal since the amount of added fat seldom exceeds 3 wt % of the diet. Some amounts of vegetable oils, fatty acid calcium salts, and mixtures of mainly saturated fatty acids (stearic and palmitic acids, for example) may be added to the diet. Since fats having a low iodine value are usually poorly digestible (the higher the iodine number, the greater the unsaturation, or the greater the number of C═C bonds present in the fat), most added fats have an iodine value much greater than 10. For example, soybean oil has an iodine value of about 120-136, and corn oil about 109-133.

Microbes in the rumen ferment carbohydrates of the feed to acetic acid, butyric acid and propionic acid, with propionic acid generally being the most important precursor of glucose. These acids may be absorbed through the rumen wall, and transported to the liver wherein they are converted to useful nutrients. Acetate may be consumed in the liver for producing energy. It may also be converted to longer fatty acids in the liver. These fatty acids may function as precursors to fat. Part of the acetate may be transferred with the blood circulation to the mammary gland, where the acetate may be used for the synthesis of fatty acids having generally sixteen or fewer carbon. Butyric acid may also be used as a precursor of milk fat.

Part of the protein in the feed generally degrades by means of microbes in the rumen to ammonia, part of which is absorbed through the rumen wall and may be converted to urea in the liver. Another part of the protein may be converted by microbes to microbial protein, which may then be absorbed from the small intestine as amino acids. Still another part of the protein in the feed may be transported directly to the small intestine and may be absorbed as amino acids, such as the protected amino acids. Under some conditions, high protein intake from the feed may lead to increased urea concentrations of milk, and does not thus necessarily increase milk protein content.

Fat in the feed may be modified by the rumen, and thus the milk fat profile may generally not be the same as the profile of fat in the feed. All fats which are not completely inert in the rumen may decrease feed intake and rumen digestibility of the feed material. Milk composition and fat quality may be influenced by the diet of the ruminant. Oil feeding (for example, vegetable oils) may have negative effects on both rumen function and milk formation. The milk protein concentration may be lowered, the fat concentration may be decreased, the proportion of trans fatty acids may be increased and the properties of the fat during industrial milk processing may be weakened. Typical milk fat may contain more than 70 wt % of saturated fatty acids, and about one third of the milk fat may be palmitic acid.

The detrimental effect of oil and fat feeding may be diminished by preventing triglyceride fat hydrolysis. Fat hydrolysis may be decreased for example by protecting fats with formaldehyde treated casein. Another alternative is to make insoluble fatty acid calcium salts whereby hydrogenation in the rumen may be avoided. However, the disadvantages of fatty acid salts limit their usability in feeds. The pungent taste of the salts generally may lead to a decreased feed intake. The salts may also interfere with pelletizing of the feed.

The nutrients obtained from the feed may be metabolized in a number of ways before forming milk components. The saturated and unsaturated fatty acids in the feed that are transported to the small intestine may be absorbed as micelles and may be converted in the small intestine wall to triglycerides, phospholipids and lipoproteins. These may be transported in the lymph, past the liver and into blood circulation for the needs of, for example, muscles and the mammary gland. Thus, any long-chain fatty acids absorbed from the diet cannot cause fatty liver. Fatty liver arises when the animal loses weight, and often occurs when metabolizing high amounts of saturated fatty acids.

Cell energy is generated in the mitochondria. Mitochondria produce energy, adenosine triphosphate (ATP), for the needs of the whole cell metabolism system. Cells, also the cells of a mammary gland, contain dozens of mitochondria. Particularly the mammary gland and the heart muscle need high amounts of energy. It has been determined that certain nutrient factors may enhance the mitochondrial function. ATP is the energy form which the cell uses for various needs. The intermediate product in ATP formation is called active acetic acid (acetyl-CoA).

A key measure in energy consumption is acetyl-CoA. Acetyl-CoA is generally obtained from carbohydrates and fats, and, in case of lack of energy, also from carbon chains of proteins, a process which is not economical. Acetyl-CoA may be obtained from carbohydrates via the pyruvic acid pathway which is important for non-ruminants but less significant in ruminants. The main source of acetyl-CoA in ruminants, in addition to the acetic acid formed in the rumen, is the β-oxidation of fatty acids. A ruminant does not use much glucose to produce acetyl-CoA. For that purpose acetate is used. The acetate is partly derived from β-oxidation of fatty acids, wherein palmitic acid provides a significant role.

It has been determined that saturated fatty acids, such as palmitic acid, from the feed may be surprisingly suitable for producing acetic acid and also acetyl-CoA. Further, when suitable enzymes and nutritional factors enhancing mitochondrial function are present, the availability of energy from the mitochondria is flexible and follows the demand. Palmitic acid is a good energetic preform wherefrom energy can “easily” be taken for use.

The saturated fat, palmitic acid, has been determined to be an important source of energy. Palmitic acid is also used in several cell functions and in functional molecules for several different tasks. Enzymes can synthesize palmitic acid, for example, in the liver and in the mammary gland. Different tissues obtain energy via β-oxidation of palmitic acid. If the eight acetyl-CoAs produced from palmitic acid are used for complete oxidation in the citric acid cycle, 129 ATP molecules may be obtained from one palmitic acid molecule. When the function of mitochondria is effective, a lot of energy may be obtained from palmitic acid whenever needed.

Energy is important for the production of milk components. Lactose (a disaccharide of glucose and galactose) may be the most important factor affecting the osmotic pressure of milk and thus lactose synthesis also regulates the amount of secreted milk. About 80-85% of the carbon of milk lactose may be derived from glucose. Part of the carbon of galactose may be produced from acetate. Lactose is synthesized in the Golgi apparatus of the cells in the mammary gland, and the process requires three ATP molecules for the formation of one lactose molecule. A sufficient supply of acetate may therefore allow for glucose to be saved for lactose production.

The amino acids needed for the synthesis of milk protein may be partly obtained from the blood. Non-essential amino acids may be synthesized in the mammary gland by utilizing the carbon C2 chain of acetate, but this process also requires ATP energy. Approximately 30 mmol ATP/1 g protein is needed in this protein synthesis. The energy needed for the synthesis of milk fat varies depending on how the milk fat is formed. Part of the fatty acids may be obtained in de novo synthesis in the mammary gland, and part may be obtained via the digestive tract from the feed, or after conversion in the rumen or in the liver. Further, esterification of fatty acids requires 10.5 mmol ATP per 1 g fat.

When fatty acids are synthesized in the udder (that is, de novo synthesis), about 27 mmol ATP per gram of fat is required. Therefore, the more milk fat components are obtained as fatty acids from the blood circulation, the more energy may be saved for other purposes. Short and middle-chain fatty acids are obtained only via de novo synthesis, and the long-chain fatty acids (C18 and longer) are obtained only from the blood circulation. Of the milk fatty acids, essentially only palmitic acid can be produced in both ways. In view of energy economy, it would be desirable to obtain more palmitic acid directly from the blood circulation.

The composition of milk fat usually differs significantly from the fat composition of the feed. Rumen hydrolysis, as well as hydrogenation, partly influence this difference. Also de novo synthesis from acetate takes place in the mammary gland and affects the composition of milk fat. In the liver, mostly the longer fatty acids, and also palmitic acid, are synthesized. In the udder on the other hand, mainly the short chain fatty acids, but also palmitic acid, are synthesized. A high concentration of fatty acids of ≧C18 in the diet may lower fatty acid synthesis.

Conventional feed raw materials may generally include common protein, carbohydrate, and/or fat containing materials used in feeds. The protein, carbohydrate and/or fat containing raw materials may include, for example, grains, peas, beans, molasses and vegetable oilseed crushes or meals. In addition to protein, carbohydrate, and fat containing raw materials, the feed may also contain other raw materials, such as minerals, additives and/or auxiliary agents. Additives may include micronutrients and vitamins. Examples of auxiliary agents may include pelletizing agents, such as lignin sulphates and/or colloidal clay. In an embodiment, a mixture of at least two of the protein, carbohydrate and/or fat containing raw materials may be used. In various embodiments, the protein content of the mixture may be about 0.1 wt % to about 55 wt %, about 5 wt % to about 45 wt %, or about 8 wt % to about 40 wt %. The protein content may be measured, for example, by using the Kjeldahl Nitrogen analysis method. In embodiments, the starch content of the mixture may be about 0.1 wt % to about 50 wt %, about 5 wt % to about 40 wt %, about 5 wt % to about 35 wt %, or about 5 wt % to about 20 wt %. The starch content may be measured, for example, by the method of AACCI 76-13.01 (American Association of Cereal Chemists International—Method 76-13.01—Total Starch Assay Procedure (Megazyme Amyloglucosidase/alpha-Amylase Method)).

It has been surprisingly determined that by a certain type of nutriment it is possible to energetically efficiently increase the proportion of milk fat derived from the feed, whereby energy is saved in the mammary gland for the synthesis of protein and lactose, and thereby milk production is increased. The most abundant fatty acid in milk is palmitic acid which can be obtained from both the blood circulation and via de novo synthesis. By configuring a nutriment appropriately, it may be possible to transfer fatty acids, via the digestive tract, into the blood circulation. In an embodiment, such a nutriment may include an ingestible carrier, and at least one fatty acid moiety covalently linked to the carrier. In an embodiment, the fatty acid may be palmitic acid. In an embodiment the carrier may be a polymer. Such a nutriment may be dispersed into drinking water or feed for consumption by the ruminant. For dispersal in liquid, an additional dispersant, such as a surfactant, for example, may be used to improve separation of the particles and prevent settling or clumping.

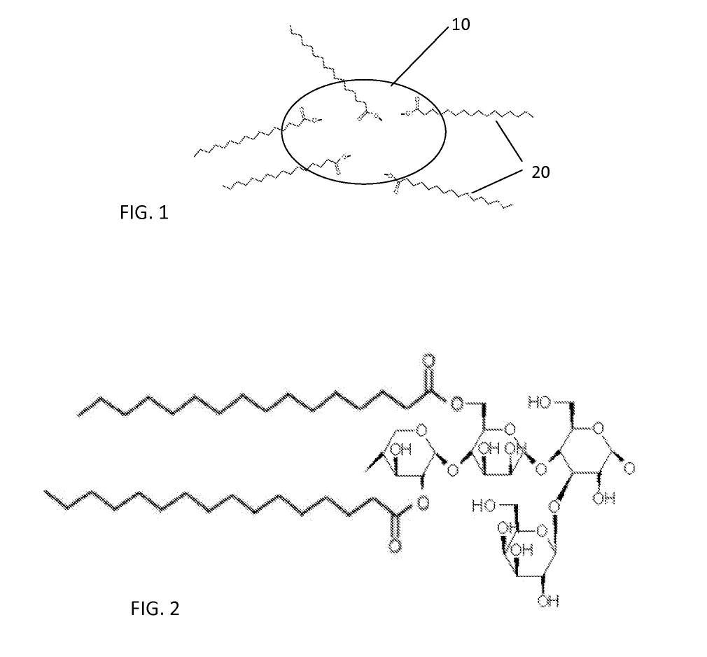

In an embodiment, a ruminant feed that increases milk production and/or the milk fat content, may include a nutritional component, at least one carrier, and at least one fatty acid moiety covalently linked to the carrier. A nutriment, or feed component may generally be represented as depicted in FIG. 1, which shows a carrier particle 10, with a plurality of fatty acid moieties 20 covalently linked thereto. A fatty acid moiety 20 may be derived from a fatty acid that has at least one functional group, and a carrier 10 may be derived from a carrier that has at least one functional group that is capable of covalently linking with a functional group of the fatty acid, to covalently link the fatty acid moiety to the carrier.

The carrier may be any of a variety of particulate materials. In various embodiments, the carrier may be feed particles, polymers, copolymers, wood particle, hay particle, grain particle, alfalfa particle, protein particle, yeast particle, corn stover particle, or combinations thereof. Some additional examples may include polysaccharides, proteins, cuticle, lignocellulose, nucleic acids, nucleotides, hemicellulose, starch, galactan, pectin, arabinogalactan, xylan, glycan, polyethylene glycol, monosaccharides, or combinations thereof.

Some examples of polymers may include carbohydrates, triglycerides, plant cuticular waxes, cutins, yeast cell wall polymers, glucans, lignans, tannins, polymerized polyphenols, proteins, chitin polymers, xylans, fructans, pullulans, or combinations thereof. Some examples of co-polymers may include plant-sourced glycoproteins (derived from plant materials).

In an embodiment, the fat component of the feed may include free, or unesterified fatty acid moieties covalently linked to the carrier. The content of saturated free fatty acids in the fat component may be at least about 90 wt %, at least about 95 wt %, at least about 97 wt %, at least about 98 wt %, at least about 99 wt %, or about 100 wt %—essentially no unsaturated fatty acids. The feed composition may be substantially free of trans fatty acid (unsaturated fatty acid having a particular isomeric configuration). “Substantially free” means, within this context, that various embodiments of a diet may contain at most about 5%, at most about 4%, at most about 3%, at most about 2%, at most about 1%, at most about 0.5%, or no trans fatty acids.

In an embodiment, the free fatty acid moiety may be a palmitic acid moiety. Further, the fatty acid moiety may consist essentially of free palmitic acid (essentially pure palmitic acid). In embodiments, the fatty acid moiety may contain at least about 90 wt %, at least about 95 wt %, at least about 98 wt %, at least about 99 wt %, or about 100 wt % palmitic acid moiety.

The fatty acid moiety may be covalently linked to the carrier through a linker, and the linker may be an ether bond, thioether bond, carbonyl bond, ester bond, imino bond, amide bond, imide bond, urethane bond, urea bond, carbonate bond, disulfide bond, maleimide bond, sulfonyl bond, sulfonate bond, phosphonyl bond, a phosphonate bond, Schiff-base bond, a bond resultant of an Amadori rearrangement, a Ugi reaction, or a Diels-Alder adduct, or combinations thereof. FIG. 2 depicts a representation of a simplified hemi-cellulose linked with palmitic acid via an ester bond.

In an embodiment, the feed may, in some cases, not contain a high amount of fatty acid salts, such as calcium salt, because of the generally negative effect such salts may have on milk production. In embodiments, there may be at most 1 wt %, at most 0.5 wt %, at most 0.1 wt %, or at most 0.02 wt % fatty acid salts in the feed. In an embodiment, there may be substantially no, or no fatty acids as salts present in the feed.

In an embodiment, the feed may, in some cases, not contain a high amount of triglycerides. In embodiments, there may be at most about 7 wt %, at most about 5 wt %, at most about 3 wt %, or at most about 1 wt % triglycerides in the feed.

In embodiments, the fatty acids may have an iodine value of at most about 4, at most about 2, at most about 1.5, or at most about 1. In embodiments, the fatty acids may have a melting point equal to or greater than about 40° C. In alternative embodiments, the fatty acids may have a melting point equal to or less than about 80° C. In further embodiments, the fatty acids may have a melting point of about 40° C. to about 80° C. Some specific examples of the melting points may be about 40° C., about 45° C., about 50° C., about 55° C., about 55° C., about 60° C., about 65° C., about 70° C., about 75° C., about 80, or ranges between any two of these values (including endpoints).

The total amount of the fatty acid in the feed may vary by feed type. As an example, the total amount of the fatty acid may be at least about 4 wt %. For example, the amount of palmitic acid moiety in the feed may be at least about 4 wt % of the total weight of the feed. In embodiments, some examples may include at least about 4 wt %, at least about 6 wt %, at least about 8 wt %, or at least about 10 wt %, and may vary between at least about 4 wt % to at most about 50 wt %. In embodiments, the lower limit for the total amount of the fatty acid in the feed may be at least about 10 wt %, at least about 12 wt %, at least about 15 wt %, and the upper limit may be at most about 35 wt %, at most about 30 wt %, or at most about 25 wt % by weight. If the feed is an energy concentrate feed, the total amount of the fatty acid may be about 15 wt % to about 25 wt %. In an embodiment of energy feed, the total amount of the fatty acid may be about 20 wt %. In a mineral concentrate feed, the amount of the fatty acid may be about 25 wt % to about 35 wt %. In an embodiment of mineral feed, the total amount of the fatty acid may be about 30 wt %. In an amino acid concentrate feed, the total amount of the fatty acid may be about 10 wt % to about 20 wt %, for example about 11 wt % to about 19 wt %. In an embodiment of amino acid feed, the total amount of the fatty acid may be about 15 wt %. In a protein concentrate feed, the total amount of the fatty acid may be about 10.5 wt % to about 20 wt %.

The feed may, in some cases, not contain other saturated free fatty acids other than those that are covalently bound to the carrier, or the feed may contain at most about 5 wt %, at most about 1 wt %, at most about 0.5 wt %, at most 0.1 wt % of the other saturated free fatty acids. The proportion of palmitic acid of the free saturated fatty acids in the feed may be at least about 90 wt %, at least about 95 wt %, at least about 97 wt %, at least about 98 wt %, at least about 99 wt %, or about 100 wt %—wherein all of the saturated free fatty acid is palmitic acid.

A feed configured as described above introduces glucose, palmitic acid and amino acids to the ruminant's metabolic system. The feed may also enhance mitochondrial function. The feed improves the degree of energy utilization in the milk production process of ruminants. When the utilization of energy is improved, milk production, that is, milk yield has been found to increase and the concentration of fat in the milk also increases. The feed intensifies fat synthesis in the mammary gland by allowing the main component of milk fat for use in the cells to be taken directly to the cells, reducing the need for synthesis by the cells, and thus saving energy. Thus, the limiting glucose may be used more effectively in lactose production whereby milk production increases. The milk protein content may also be increased if there is no need to produce glucose from amino acids, which may also be obtained directly from the feed. Such a feed may result in a reduction of weight loss at the beginning of the lactation season, and thus fertility problems may also be decreased.

It has now been determined that when animals obtain ATP energy from the acetate formed in the rumen and acetate oxidized from palmitic acid, obtain glucose from a glucogenic feed, and obtain amino acids from proteinaceous feed, both the amount of milk and the protein and fat content of the milk can be increased. The palmitic acid therefore may be obtained from such a source of fat, and in such a way that it does not disturb the rumen, worsen the digestibility of roughage, and decrease eating (feed intake).

In an embodiment, a feed that contains a fatty acid moiety wherein at least about 90 wt % of the fatty acid is palmitic acid, improves milk yield, increases milk fat content (% by weight) and may also increase milk protein content (% by weight).

The feed may additionally contain one or more nutritional components selected from the group consisting of carbohydrate sources, nitrogen sources, amino acids, amino acid derivatives, minerals, vitamins, antioxidants, glucogenic precursors and/or components which enhance mitochondrial function.

A surprisingly large increase in milk production may be obtained when cows are fed a feed which contains a combination of palmitic acid, a glucogenic precursor, amino acids and certain components which intensify cell level function (that is, mitochondria function enhancing components). It has been determined that the addition of palmitic acid as discussed herein, together with suitable feed components, provides for improved energy efficiency of ruminant feeding and feed utilization. In an embodiment, a feed may contain palmitic acid that is completely inert in the rumen and the utilization of which in the ruminant's metabolic system is affected or surprisingly improved by the preparation process and suitable components of the feed.

Consequently, in an embodiment, the feed may include at least one glucogenic precursor. The glucogenic precursor may be selected from the group consisting of glycerol, propylene glycol, molasses, propionate, glycerine, propane diol, calcium propionate, steam-exploded sawdust, steam-exploded wood chips, steam-exploded wheat straw, algae, algae meal, microalgae, or combinations thereof. In embodiments, the amount of the glucogenic precursor in the feed may be about 1 wt % to about 20 wt %, or about 5 wt % to about 15 wt %.

Further, the feed may also contain added amino acids and/or amino acid derivatives. The added amino acids or derivatives may be amino acids or derivatives selected from the group consisting of the essential amino acids leucine, lysine, histidine, valine, arginine, threonine, isoleucine, phenylalanine, methionine, tryptophan, or combinations thereof. In an embodiment, non-essential amino acids or derivatives may also be added, and may include alanine, asparagine, aspartate, cysteine, glutamate, glutamine, glycine, proline, serine, tyrosine, or combinations thereof. The amount of added amino acids in the feed may be about 0.1 wt % to about 2 wt %, or about 0.5 wt % to about 1 w %.

In embodiments, the feed may include added components that enhance the function of mitochondria. Mitochondrial function enhancing components may be selected from the group consisting of carnitine, biotin, other B vitamins, omega-3-fatty acids, ubiquinone and combinations thereof. In embodiments, the amount of the mitochondrial function enhancing components may be about 0.5 wt % to about 5 wt %, or about 1 wt % to about 3 wt %.

In embodiments, the carbohydrate source may be selected from the group consisting of microalgae, sugar beet pulps, sugar canes, wheat bran, oat hulls, grain hulls, soybean hulls, peanut hulls, wood, brewery byproduct, beverage industry by-products, forages, roughages, molasses, sugars, starch, cellulose, hemicellulose, wheat, corn, oats, sorghum, millet, barley, barley fibre, barley hulls, barley middlings, barley bran, malting barley screenings, malting barley and fines, malt rootlets, maize bran, maize middlings, maize cobs, maize screenings, maize fibre, millet, rice, rice bran, rice middlings, rye, triticale, brewers grain, coffee grinds, tea leaf ‘fines’, citrus fruit pulp, rind residues, or combinations thereof.

In embodiments, the nitrogen source may be selected from the group consisting of microalgae, oilseed meals, soy meals, bean meals, rapeseed meals, sunflower meals, coconut meals, olive meals, linseed meals, grapeseed meals, distiller dry grains solids, camelina meal, camelina expeller, cotton seed meal, cotton seed expeller, linseed expeller, palm meal, palm kernel meal, palm expeller, rapeseed expeller, potato protein, olive pulp, horse beans, peas, wheat germ, corn germ, corn germ pressed fiber meal residue, corn germ protein meal, whey protein concentrate, milk protein slurries, milk protein powders, animal protein, or combinations thereof.

In embodiments, the mineral may be a salt of Ca, Na, Mg, P, K, Mn, Zn, Se, Cu, I, Fe, Co, Mo, or combinations thereof. These minerals may be provided using any of a number of mineral sources. In general, any GRAS (generally recognized as safe) mineral source may be used which provides a bioavailable mineral. Some examples include copper sulphate, sodium selenite, selenium yeast, and chelated minerals. Table 1 shows some examples of suitable mineral sources.

TABLE 1
GRAS Mineral Sources

| | | | |
|---|---|---|---|
| Calcium Acetate | Calcium Carbonate | Calcium Chloride | Calcium Gluconate |
| Calcium Hydroxide | Calcium Iodate | Calcium | Calcium Oxide |
| | | Iodobehenate | |
| Calcium Sulfate | Cobalt Acetate | Cobalt Carbonate | Cobalt Chloride |
| (anhydrous or | | | |
| dihydrate) | | | |
| Cobalt Oxide | Cobalt Sulfate | Dicalcium | Magnesium Acetate |
| | | Phosphate | |
| Magnesium Carbonate | Magnesium Oxide | Magnesium Sulfate | Manganese Carbonate |
| Manganese Chloride | Manganese Citrate | Manganese | Manganese |
| | (soluble) | Gluconate | Orthophosphate |
| Manganese Oxide | Manganese | Manganese Sulfate | Monocalcium |
| | Phosphate | | Phosphate |
| | (dibasic) | | |
| Monosodium | Potassium Acetate | Potassium | Potassium Carbonate |
| Phosphate | | Bicarbonate | |
| Potassium chloride | Potassium Iodate | Potassium Iodide | Potassium Sulfate |
| Sodium Acetate | Sodium Chloride | Sodium Bicarbonate | Disodium Phosphate |
| Iron Ammonium | Iron Carbonate | Iron Chloride | Iron Gluconate |
| Citrate | | | |
| Iron Oxide | Iron Phosphate | Iron Pyrophosphate | Iron Sulfate |
| Reduced Iron | Sodium Iodate | Sodium Iodide | Sodium |
| | | | Tripolyphosphate |
| Sodium Sulfate | Tricalcium | Zinc Acetate | Zinc Carbonate |
| | Phosphate | | |
| Zinc Chloride | Zinc Oxide | Zinc Sulfate | |

In embodiments, the vitamin may be selected from the group consisting of vitamin A, vitamin C, vitamin D, vitamin E, vitamin K, vitamin B1, vitamin B2, pantothenic acid, niacin, biotin, choline, or combinations thereof.

In embodiments, the antioxidant may be selected from the group consisting of alpha-carotene, beta-carotene, ethoxyquin, BHA, BHT, cryptoxanthin, lutein, lycopene, zeaxanthin, vitamin A, vitamin C, vitamin E, selenium, alpha-lipoic acid, or combinations thereof.

The feed may essentially be any type of feed, and may include any compound feed (industrially produced mixed feed) intended for feeding of a lactating animal. Some examples may include complete feeds (compound feed containing all main nutrients except nutrients obtained from roughage), and concentrate feeds, such as protein concentrate feeds, mineral concentrate feeds, energy concentrate feeds, and amino acid concentrate feeds. The energy concentrate feeds, amino acid concentrate feeds, and mineral concentrate feeds may provide better results. The term “concentrate feed” generally refers to a compound feed which has a high concentration of the indicated substances. Typical concentrate feed are used in combination with other feed, such as grains. Various embodiments of concentrated feed as described herein may include more nutrients than conventional supplements.

In embodiments, a complete feed may contain about 15 wt % to about 50 wt %, about 16 wt % to about 40 wt %, or about 17 wt % to about 35 wt % protein and/or amino acids and/or peptides. This amount may include mainly proteins, but also may include peptides and small amounts of free amino acids. The amino acid and/or protein and/or peptide content may be measured, for example, by using the Kjeldahl Nitrogen analysis method. In an embodiment, an amino acid concentrate feed or protein concentrate feed may contains about 20 wt % to about 40 wt %, or about 24 wt % to about 35 wt % amino acids and/or protein. In embodiments, a mineral concentrate feed may contain less than about 25 wt %, or less than about 20 wt % of amino acids and/or protein. In embodiments, an energy concentrate feed may contain about 5 wt % to about 50 wt %, or about 8 wt % to about 40 wt % amino acids and/or protein.

In an embodiment, a complete feed may contain about 4 wt % to about 50 wt %, about 6 wt % to about 45 wt %, about 8 wt % to about 40 wt %, or about 12 wt % to about 35 wt % starch. The starch content may be measured, for example, by the AACCI 76-13.01 method. An amino acid concentrate feed or protein concentrate feed may contain about 1 wt % to about 30 wt %, about 5 wt % to about 20 wt % starch. A mineral concentrate feed may contain less than about 20 wt %, or less than about 15 wt % starch. An energy concentrate feed may contain about 5 wt % to about 50 wt %, or about 5 wt % to about 40 wt % starch.

Palmitic acid has a melting point of about 63° C. and an iodine value less than or equal to about 1. Palmitic acid therefore essentially does not disturb rumen function since palmitic acid essentially completely passes through the rumen and does not decrease feed intake like fatty acid calcium salts or fatty acids which have a lower melting point and a higher iodine value.

A process for producing a ruminant nutriment containing fatty acids covalently linked to carrier particles may include covalently bonding at least one fatty acid moiety to a carrier to produce fatty acid particles, and dispersing the fatty acid particles in at least one of ruminant feed and drinking water for ingestion by the ruminant. For a feed, the dispersing may include mixing the fatty acid particles with ruminant feed to produce a feed mixture, wherein the fatty acid moiety is present in the feed mixture at a concentration of at least about 4 wt %. In an embodiment, the fatty acid may be palmitic acid.

After mixing the fatty acid particles with ruminant feed, the mixture may be extruded, or pelletized, or processed in other ways to produce a feed product which may be taken orally by the ruminant breed to which it is to be fed. Prior to, or during mixing, additional nutritional components, such as a carbohydrate source, a nitrogen source, an amino acid, an amino acid derivative, a mineral, a vitamin, an antioxidant, a glucogenic precursor, or combinations thereof, may also be added to the feed mixture.

In various embodiments, the degree of utilization of the feed may be increased by about 5%, or about 10%, or about 15% when calculated as the efficiency of utilization of metabolizable energy intake for milk production (kl). Unlike several other high-fat feeds, a feed according to an embodiment as described herein may also be palatable, even highly attractive for ruminants, for example, cows. Embodiments of the feed may not necessarily therefore result in a decrease in feed intake as compared to a feed which does not contain added fats (the fat percentage of a feed that does not contain added fats may typically be about 2 wt % to about 4 wt %).

In an embodiment, a feed may be configured as an energy concentrate feed or a mineral concentrate feed, and may contain in addition to palmitic acid covalently bound with a carrier, a glucose source, and at least one of propylene glycol, glycerol and salts of propionic acid (sodium, calcium). The energy concentrate feed or mineral concentrate feed may also contain small amounts of mitochondrial function enhancing components, such as carnitine, biotin, other B vitamins, omega-3 fatty acids, ubiquinone, and combinations thereof. These concentrate feeds may also contain added amino acids. An amount of palmitic acid in an energy concentrate feed may be between about 15 wt % to about 25 wt %, and in an embodiment may be about 20 wt %. In a mineral concentrate feed the content of palmitic acid may be about 25 wt % to about 35 wt %, and in an embodiment, may be about 30 wt %.

In an embodiment, the feed may be an amino acid concentrate feed that contains, in addition to palmitic acid covalently bound with a carrier, glucose sources (a glucogenic precursor) and also amino acids. The nutrients in the feed may be utilized more effectively only after the cell level energy metabolism has been intensified with the aid of palmitic acid. The added amino acids may include methionine, lysine or histidine, or any combination thereof. In one embodiment the amino acid concentrate feed may also contain components enhancing mitochondrial function, especially as regards beta oxidation and fat synthesis. Such components may include for example carnitine, biotin, other B vitamins, omega-3-fatty acids, ubiquinone, and combinations thereof. An amount of palmitic acid in an amino acid concentrate feed may be about 10 wt % to about 20 wt %, and in an embodiment, may be about 15 wt %.

A feed prepared and configured as discussed herein may be fed to a ruminant, or provided to the ruminant for ingestion, whereby ingestion of the feed can deliver a daily amount of fatty acid. In embodiments, the daily amount of fatty acid may be about 0.2 kg/day to about 1.0 kg/day, or about 0.3 kg/day to about 0.8 kg/day, or about 0.4 kg/day to about 0.7 kg/day. Some specific examples of the daily amount of the fatty acid may be about 0.2 kg/day, about 0.3 kg/day, about 0.4 kg/day, about 0.5 kg/day, about 0.6 kg/day, about 0.7 kg/day, about 0.8 kg/day, about 0.9 kg/day, about 1.0 kg/day, or ranges between any two of these values (including endpoints).

The delivery can also be expressed as an amount of fatty acid ingested via the feed per amount of produced milk. In embodiments, the amounts may be configured to provide about 1 g to about 30 g fatty acid per kg milk/day, about 6 g to about 16 g fatty acid/kg milk/day, or about 10 g fatty acid/kg milk/day. These daily amounts or amounts per 1 kg milk production can suitably be applied in any method or use disclosed herebelow. The daily amounts disclosed above may be the amounts of free palmitic acid.

In accordance with the discussion presented herein, a method for increasing milk production of a lactating animal and/or increasing the concentrations of protein and fat in milk is also provided. The method includes feeding a ruminant an amount of a feed configured as presented herein. The feed is provided to the ruminant for ingestion.

A method for increasing milk fat content and/or for increasing milk production includes giving a lactating ruminant a milk fat increasing amount and/or a milk volume increasing amount of a feed configured as discussed herein. The feed is provided to the ruminant for ingestion.

A method of increasing the milk protein content includes giving a lactating ruminant a milk protein increasing amount of a feed configured as discussed herein. The feed is provided to the ruminant for ingestion.

Methods may optionally further include recovering the milk produced by a lactating ruminant to which a feed configured as discussed herein is fed.

A method for using palmitic acid for preparing a ruminant feed includes providing an amount of added palmitic acid that is at least about 4 wt %, and, during the preparation process, covalently bonding the palmitic acid with an ingestible carrier particle.

A method for using palmitic acid for increasing milk production of a lactating animal and/or for increasing concentrations of protein and fat in milk includes giving a lactating animal one or more feeds which provide the animal with a daily amount of palmitic acid. For example, the daily amount can be about 0.2 kg/day to about 1.0 kg/day, or about 0.3 kg/day to about 0.8 kg/day, or about 0.4 kg/day to about 0.7 kg/day. Some specific examples of the daily amount of the fatty acid may be about 0.2 kg/day, about 0.3 kg/day, about 0.4 kg/day, about 0.5 kg/day, about 0.6 kg/day, about 0.7 kg/day, about 0.8 kg/day, about 0.9 kg/day, about 1.0 kg/day, or ranges between any two of these values. All other features of embodiments disclosed herein for the feeds are applicable for the disclosed uses.

In embodiments, a ruminant compound feed may include:
total lipids that may be in an amount of about 10.1 wt % to about 57 wt %, or about 10.5 wt % to about 45 wt %, or about 10.5 wt % to about 40 wt %, or about 10.5 wt % to about 30 wt %, or about 10.5 wt % to about 20 wt %, or about 11 wt % to about 14 wt %;
free palmitic acid that may be in an amount of about 10.1 wt % to about 50 wt %, or about 10.1 wt % to about 35 wt %, or about 10.1 wt % to about 25 wt %;
proteins that may be in an amount of about 15 wt % to about 50 wt %, or about 16 wt % to about 40 wt %, or about 17 wt % to about 35 wt %; and
starch in an amount of about 4 wt % to about 50 wt %, or about 6 wt % to about 45 wt %, or about 8 wt % to about 40 wt %, or about 12 wt % to about 35 wt %; and
   the amount of free palmitic acid may be at least about 40 wt %, or at least about 45 wt %, or at least about 50 wt %, or at least about 55 wt %, or at least about 60 wt %, or at least about 65 wt %, or at least about 70 wt %, or at least about 75 wt %, or at least about 80 wt %, or at least about 85 wt %, or at least about 90 wt % of the total lipids.

The ruminant compound feed may be a complete feed, and may be in the form of pellets or granules. The ruminant compound feed may additionally include at least one component selected from the group consisting of a glucogenic precursor, for example, in an amount of about 1 wt % to about 20 wt %, or about 5 wt % to about 15 wt %; a mitochondrial function enhancing component, for example in an amount of about 0.5 wt % to about 5 wt %, or about 1 wt % to about 3 wt %; and amino acids, for example, in an amount of about 0.1 wt % to about 6 wt %, or about 1.5 wt % to about 3 wt %.

The ruminant compound feed may be obtainable by adding a fatty acid/carrier component, wherein at least about 90% of the fatty acid is palmitic acid, to conventional feed raw materials.

### Example 1

### Fatty Acid/Carrier Particles

Palmitic acid is covalently bound to hemicellulose to provide a fatty acid-carrier particle as generally represented in FIG. 2. Hemicellulose has a large number of free hydroxyl groups and therefore attachment of palmitic acid can be performed using an esterification reaction of the hydroxyl groups with the carboxylic acid functional group of the fatty acid. Ester bonds are produced when carboxylic acids are heated with alcohols (hydroxyls) in the presence of an acid catalyst, as represented by the following:

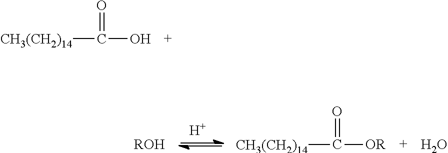

wherein R represent a hemicellulose molecule and ROH represents one of a plurality of hydroxyl sites on a hemicellulose molecule. Each resultant particle includes a plurality of covalently bonded palmitic acid moieties.

### Example 2

### Ruminant Feed

The following three compositions are examples of feed compositions (in wt %) that increase blood glucose and blood palmitic acid supply for the mammary gland.

| Composition # | 1 | 2 | 3 |
|---|---|---|---|
| Feed grain (wheat, barley, oats) | 0-50 | 10-40 | 20-30 |
| Sugar beet pulp | 0-30 | 5-25 | 10-20 |
| Wheat bran | 0-30 | 5-25 | 10-20 |
| Molasses | 0-8 | 1-5 | 1-3 |
| Protein crush (rapeseed, soya) | 0-50 | 10-40 | 20-30 |
| Wheat middlings | 0-20 | 5-15 | 8-12 |
| Minerals | 0-5 | 1-4 | 2-3 |
| Premixes (vitamins, mineral nutrients) | 0-2 | 0.5-1.5 | 0.8-1.2 |
| Propylene glycol | 1-15 | 4-14 | 8-12 |
| Glycerol/Sodium propionate | 0-5 | 1-4 | 2-3 |
| Amino acid mixture | 0-2 | 0.1-1.5 | 0.3-0.9 |
| B vitamin mixture | 0-2 | 0.5-1.5 | 0.8-1.2 |
| Carnitine | 0-1 | 0.1-0.8 | 0.2-0.6 |
| Palmitic acid | 10.1-30 | 12-25 | 15-22 |

(wherein the palmitic acid is a component of particles of Example 1)

### Example 3

### Two-Month Study Confirming Efficacy of Fatty Acid/Carrier Particles in Dairy Cow Feed

A feeding experiment is performed for about two months where a conventional complete feed is replaced by a feed having the following composition (% by weight):

| | |
|---|---|
| Sugar beet pulp | 20 |
| Barley | 20 |
| Palmitic acid | 20 (as a component of particles of Example 1) |
| Wheat bran | 14 |
| Oat bran | 10 |
| Propylene glycol | 10 |
| Molasses | 2 |
| Sodium bicarbonate | 2 |
| Biotin | 1 |
| Carnitine premix | 0.4 |
| Methionine premix | 0.5 |

The above test feed is given to one set of cows, and a standard conventional complete feed will be given to a second set of cows as a reference. The following results are obtained after the two-month study:

| | Reference | Test feed |
|---|---|---|
| Milk kg/d | 29.5 | 32.5 |
| Fat wt % | 3.98 | 4.43 |
| Protein wt % | 3.15 | 3.37 |

The results for a feed with the formulation as provided above show that the milk production as well as fat and protein concentrations increase significantly. In addition, the degree of feed utilization, measured as the efficiency of utilization of metabolizable energy intake for milk production (kl), also significantly improve.

This disclosure is not limited to the particular systems, devices and methods described, as these may vary. The terminology used in the description is for the purpose of describing the particular versions or embodiments only, and is not intended to limit the scope.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or an (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A feed composition for ruminants, the feed composition comprising:
at least one carrier;
at least one fatty acid moiety covalently linked to the carrier; and
at least one nutritional component.

2. The feed composition of claim 1, wherein the fatty acid moiety is derived from a fatty acid, and the at least one carrier is derived from a carrier comprising at least one functional group capable of binding with a functional group of the fatty acid to covalently link the fatty acid moiety with the carrier.

3. The feed composition of claim 1, wherein the carrier is at least one feed particle.

4. The feed composition of claim 1, wherein the carrier is at least one wood particle, hay particle, grain particle, alfalfa particle, plant stalk particle, plant stem particle, soybean straw particle, protein particle, yeast particle, corn stover particle, or combinations thereof.

5. The feed composition of claim 1, wherein the carrier is at least one polymer.

6. The feed composition of claim 5, wherein the at least one polymer is a carbohydrate, triglyceride, plant cuticular wax, cutin, yeast cell wall polymer, glucan, lignan, tannin, polymerized polyphenol, protein, chitin polymer, xylan, fructan, pullulan, or combination thereof.

7. The feed composition of claim 1, wherein the carrier is at least one co-polymer.

8. The feed composition of claim 7, wherein the at least one co-polymer is a plant-sourced glycoprotein.

9. The feed composition of claim 1, wherein the carrier is a polysaccharide, a protein, cuticle, lignocellulose, a nucleic acid, a nucleotide, hemicellulose, polyethylene glycol, or combinations thereof.

10. The feed composition of claim 1, wherein the carrier is starch, galactan, pectin, glycan, arabinogalactan, xylan, a monosaccharide, or combinations thereof.

11. The feed composition of claim 1, wherein the fatty acid moiety is covalently linked to the carrier through a linker, wherein the linker comprises an ether bond, thioether bond, carbonyl bond, ester bond, imino bond, amide bond, imide bond, urethane bond, urea bond, carbonate bond, disulfide bond, maleimide bond, sulfonyl bond, sulfonate bond, phosphonyl bond, a phosphonate bond, Schiff-base bond, Amadori rearrangement bond, Ugi reaction bond, a bond resultant of a Diels-Alder rearrangement, or combinations thereof.

12. The feed composition of claim 1, wherein the fatty acid moiety comprises at least about 90% palmitic acid moiety.

13. The feed composition of claim 1, wherein the fatty acid moiety is a palmitic acid moiety.

14. The feed composition of claim 13, wherein the palmitic acid moiety is linked to the carrier through an ester bond.

15. The feed composition of claim 13, wherein the palmitic acid moiety is present in the feed composition at a concentration of at least about 4 wt %.

16. The feed composition of claim 13, wherein the palmitic acid moiety is present in the feed composition at a concentration of at least about 10 wt %.

17. The feed composition of claim 13, wherein the feed composition contains less than about 5 wt % trans-fatty acid.

18. The feed composition of claim 13, wherein the feed composition contains substantially no trans-fatty acid.

19. The feed composition of claim 13, wherein the feed composition does not contain trans-fatty acid.

20. The feed composition of claim 1, wherein the nutritional component comprises at least one of a carbohydrate source, a nitrogen source, an amino acid, an amino acid derivative, a mineral, a vitamin, an antioxidant, and a glucogenic precursor.

21. The feed composition of claim 20, wherein the carbohydrate source comprises microalgae, sugar beet pulps, sugar canes, wheat bran, oat hulls, grain hulls, soybean hulls, peanut hulls, wood, brewery by-product, beverage industry by-products, forages, roughages, molasses, sugars, starch, cellulose, hemicellulose, wheat, corn, oats, sorghum, millet, barley, barley fiber, barley hulls, barley middlings, barley bran, making barley screenings, making barley and fines, malt rootlets, maize bran, maize middlings, maize cobs, maize screenings, maize fiber, millet, rice, rice bran, rice middlings, rye, triticale, brewers grain, coffee grinds, tea leaf ‘fines’, citrus fruit pulp, rind residues, or combinations thereof.

22. The feed composition of claim 20, wherein the nitrogen source comprises microalgae, soy meals, bean meals, rapeseed meals, sunflower meals, coconut meals, olive meals, linseed meals, grapeseed meals, distiller dry grains solids, camelina meal, camelina expeller, cotton seed meal, cotton seed expeller, linseed expeller, palm meal, palm kernel meal, palm expeller, rapeseed expeller, potato protein, olive pulp, horse beans, peas, wheat germ, corn germ, corn germ pressed fiber meal residue, corn germ protein meal, whey protein concentrate, milk protein slurries, milk protein powders, animal protein, or combinations thereof.

23. The feed composition of claim 20, wherein the amino acid comprises leucine, lysine, histidine, valine, arginine, threonine, isoleucine, phenylalanine, methionine, tryptophan, alanine, asparagine, aspartate, cysteine, glutamate, glutamine, glycine, proline, serine, tyrosine, or combinations thereof.

24. The feed composition of claim 20, wherein the amino acid derivative comprises a leucine derivative, a lysine derivative, a histidine derivative, a valine derivative, an arginine derivative, a threonine derivative, an isoleucine derivative, a phenylalanine derivative, a methionine derivative, a tryptophan derivative, an alanine derivative, an asparagine derivative, an aspartate derivative, a cysteine derivative, a glutamate derivative, a glutamine derivative, a glycine derivative, a proline derivative, a serine derivative, a tyrosine derivative, or combinations thereof.

25. The feed composition of claim 20, wherein the mineral comprises salts of Ca, Na, Mg, P, K, Mn, Zn, Se, Cu, I, Fe, Co, Mo, or combinations thereof.

26. The feed composition of claim 20, wherein the vitamin comprises vitamin A, vitamin C, vitamin D, vitamin E, vitamin B1, vitamin B2, vitamin K, vitamin Q, Pantothenic acid, niacin, biotin, choline, carnitine, or combinations thereof.

27. The feed composition of claim 20, wherein the glucogenic precursor comprises glycerol, propylene glycol, molasses, propionate, glycerine, propane diol, calcium propionate, steam-exploded sawdust, steam-exploded wood chips, steam-exploded wheat straw, or combinations thereof.

28. The feed composition of claim 20, wherein the antioxidant comprises alpha-carotene, beta-carotene, ethoxyquin, BHA, BHT, cryptoxanthin, lutein, lycopene, zeaxanthin, vitamin A, vitamin C, vitamin E, selenium, alpha-lipoic acid, or combinations thereof.

29. The feed composition of claim 1, wherein:
the carrier comprises at least one of polysaccharides, proteins, lignocellulosics, nucleic acids, hemicelluloses, and polyethylene glycol;
the fatty acid moiety comprises at least one palmitic acid moiety and is linked to the polymer carrier through an ester bond;
the palmitic acid moiety is present in the feed composition at a concentration of at least about 10 wt %;
the feed composition contains substantially no trans-fatty acid; and
the nutritional component comprises a carbohydrate source, a nitrogen source, an amino acid, an amino acid derivative, a mineral, a vitamin, an antioxidant, a glucogenic precursor, or combinations thereof.

30. A nutriment for ruminants, the nutriment comprising:
an ingestible polymer carrier; and
at least one palmitic acid moiety covalently linked to the polymer carrier.

31. The nutriment of claim 30, wherein the nutriment is configured for being dispersed in drinking water.

32. The nutriment of claim 31, further comprising a dispersant for dispersing the nutriment in drinking water.

33. The nutriment of claim 30, wherein the nutriment is configured for being dispersed in feed.

34. The nutriment of claim 30, wherein the palmitic acid moiety is covalently linked to the polymer carrier through a linker, wherein the linker comprises an ether bond, a thioether bond, a carbonyl bond, an ester bond, an imino bond, an amide bond, an imide bond, a urethane bond, a urea bond, a carbonate bond, a sulfonyl bond, a sulfonate bond, a phosphonyl bond, a phosphonate bond, a Schiff-base bond, an Amadori rearrangement bond, a Ugi reaction bond, a bond resultant of a Diels-Alder rearrangement, or combinations thereof.

35. The nutriment of claim 30, wherein the palmitic acid moiety is covalently linked to the polymer carrier through an ester bond.

36. The nutriment of claim 30, wherein the polymer carrier comprises at least one of: polysaccharides, proteins, lignocellulosics, nucleic acids, hemicelluloses, and polyethylene glycols.

37. The nutriment of claim 30, wherein the palmitic acid moiety is present in the nutriment at a concentration of at least about 4 wt %.

38. The nutriment of claim 30, wherein the palmitic acid moiety is present in the nutriment at a concentration of at least about 10 wt %.

39. The nutriment of claim 30, wherein the nutriment contains less than about 5% trans-fatty acid.

40. The nutriment of claim 30, wherein the nutriment substantially does not contain trans-fatty acid.

41. The nutriment of claim 30, wherein the nutriment does not contain trans-fatty acid.

42. The nutriment of claim 30, further comprising at least one feed component selected from the group consisting of carbohydrate sources, nitrogen sources, amino acids, amino acid derivatives, minerals, vitamins, antioxidants, and glucogenic precursors.

43. A method for producing a nutriment for ruminants, the method comprising:
covalently bonding at least one palmitic acid moiety to a carrier to produce palmitic acid particles; and
dispersing the palmitic acid particles in at least one of ruminant feed and drinking water for ingestion by the ruminant.

44. The method of claim 43, wherein the dispersing comprises mixing the palmitic acid particles with ruminant feed to produce a feed mixture, wherein the palmitic acid moiety is present in the feed mixture at a concentration of at least about 4 wt %.

45. The method of claim 43, further comprising extruding the feed mixture, pelletizing the feed mixture, or both.

46. The method of claim 43, wherein the palmitic acid moiety is present in the feed mixture at a concentration of at least about 10 wt %.

47. The method of claim 43, wherein the feed mixture substantially does not contain trans-fatty acids.

48. The method of claim 44, wherein the mixing comprises mixing the palmitic acid particles with a carbohydrate source, a nitrogen source, an amino acid, an amino acid derivative, a mineral, a vitamin, an antioxidant, a glucogenic precursor, or combinations thereof.

49. The method of claim 43, wherein the carrier is at least one polymer or at least one co-polymer.

50. The method of claim 43, wherein the carrier is a polysaccharide, a protein, lignocellulose, a nucleic acid, hemicellulose, polyethylene glycol, or combinations thereof.

51. A method for increasing at least one of an amount of milk produced by a lactating ruminant and a milk fat content in the milk produced by the lactating ruminant, the method comprising feeding the lactating ruminant a feed composition comprising at least one palmitic acid moiety covalently bound to a carrier.

52. The method of claim 51, wherein the feeding of the lactating ruminant comprises providing to the lactating ruminant an amount of the feed composition to provide the lactating ruminant with a daily amount of about 0.2 kg to about 1 kg palmitic acid moiety.

53. The method of claim 51, wherein the feeding of the lactating ruminant comprises:
determining an average amount of milk produced per day for the lactating ruminant; and
providing to the lactating ruminant an amount of the feed composition to provide the lactating ruminant with a daily amount of about 1 g to about 30 g fatty acid per kg milk produced per day.

54. The method of claim 51, wherein the feeding of the lactating ruminant comprises:
determining an average amount of milk produced per day for the lactating ruminant; and
providing to the lactating ruminant an amount of the feed composition to provide the lactating ruminant with a daily amount of about 10 g fatty acid per kg milk produced per day.

55. The method of claim 51, wherein the palmitic acid moiety is present in the feed mixture at a concentration of at least about 10 wt %.

56. The method of claim 51, wherein the feed mixture substantially does not contain trans-fatty acids.

57. The method of claim 51, wherein the carrier is at least one polymer or at least one co-polymer.

58. The method of claim 51, wherein the carrier is a polysaccharide, a protein, lignocellulose, a nucleic acid, hemicellulose, polyethylene glycol, or combinations thereof.
